(19) United States
(12) Patent Application Publication
(10) Pub. No.: US 2024/0382308 A1
(43) Pub. Date: Nov. 21, 2024

(54) Variable Stiffness Delivery System for Edge-To-Edge Transcatheter Valve Repair and Methods of Making and Using Same

(71) Applicant: Evalve, Inc., Santa Clara, CA (US)

(72) Inventors: Dylan T. Van Hoven, San Carlos, CA (US); Chad J. Abunassar, Alameda, CA (US)

(73) Assignee: Evalve, Inc., Santa Clara, CA (US)

(21) Appl. No.: 18/627,545

(22) Filed: Apr. 5, 2024

Related U.S. Application Data
(60) Provisional application No. 63/503,208, filed on May 19, 2023.

(51) Int. Cl. A61F 2/24

(52) U.S. Cl. CPC A61F 2/2466; A61F 2/246; A61F 2/2463

(57) ABSTRACT

An interventional tool includes an outer sheath, a steerable sleeve disposed within the outer sheath and translatable relative thereto, an inner conduit disposed within the steerable sleeve and translatable relative thereto, and a stiffness-varying element disposed within the outer sheath and configured and arranged to transition the interventional tool between a first state having a first stiffness and a second state having a second stiffness, the first stiffness being greater than the second stiffness.

### CROSS-REFERENCE TO RELATED APPLICATIONS

This application claims priority to U.S. Ser. No. 63/503,208, filed May 19, 2023, the content of which is hereby incorporated by reference in its entirety as if fully set forth herein.

### BACKGROUND

Mitral valve regurgitation may be characterized by retrograde flow from the left ventricle of a heart through a compromised mitral valve into the left atrium. During a normal cycle of heart contraction (systole), the mitral valve ideally acts as a one-way valve to prevent flow of oxygenated blood back into the left atrium. In this way, the oxygenated blood is pumped into the aorta through the aortic valve. Valve regurgitation may significantly decrease the pumping efficiency of the heart, placing the patient at risk of severe, progressive heart failure.

Mitral valve regurgitation can result from a number of different mechanical defects in the mitral valve or the left ventricular wall. The valve leaflets, the valve chordae which connect the leaflets to the papillary muscles, the papillary muscles or the left ventricular wall may be damaged or otherwise dysfunctional. Commonly, the valve annulus may be damaged, dilated, or weakened limiting the ability of the mitral valve to close adequately against the high pressures of the left ventricle.

Common treatments for mitral valve regurgitation rely on valve replacement or repair including leaflet and annulus remodeling, the latter generally referred to as valve annuloplasty. Another technique for mitral valve repair which relies on suturing adjacent segments of the opposed valve leaflets together is referred to as the “bow-tie” or “edge-to-edge” technique. While all these techniques can be very effective, they usually rely on open heart surgery where the patient's chest is opened, typically via a sternotomy, and the patient placed on cardiopulmonary bypass. The need to both open the chest and place the patient on bypass is traumatic and has associated high mortality and morbidity.

Alternatively, mitral valve regurgitation may be corrected by transcatheter delivery of an implant that facilitates full closure of the mitral valve during each heart contraction cycle. Transcatheter delivery can be a complicated process requiring close attention and many inputs and manipulations from an implanter, interventionalist, or physician, which will collectively be referred to with the term “physician” in the remainder of this disclosure. In some cases, the stiffness of a delivery catheter may affect the ability to navigate the catheter through the tortuous anatomy, and confirm proper implantation of the implant.

### BRIEF SUMMARY

In some examples, an interventional tool includes an outer sheath, a steerable sleeve disposed within the outer sheath and translatable relative thereto, an inner conduit disposed within the steerable sleeve and translatable relative thereto, and a stiffness-varying element disposed within the outer sheath and configured and arranged to transition the interventional tool between a first state having a first stiffness and a second state having a second stiffness, the first stiffness being greater than the second stiffness.

In some examples, a method of actuating a medical device includes providing an interventional tool including an outer sheath, a steerable sleeve disposed within the outer sheath and translatable relative thereto, an inner conduit disposed within the steerable sleeve and translatable relative thereto, and a stiffness-varying element disposed within the outer sheath, and transitioning the interventional tool between a first state having a first stiffness and a second state having a second stiffness, the first stiffness being greater than the second stiffness.

### BRIEF DESCRIPTION OF THE DRAWINGS

FIG. 1 illustrates the left ventricle and left atrium of the heart during systole.

FIG. 2A illustrates free edges of leaflets in normal coaptation, and FIG. 2B illustrates the free edges in regurgitative coaptation.

FIG. 3A-3C illustrate grasping of the leaflets with a fixation device, inversion of the distal elements of the fixation device and removal of the fixation device, respectively.

FIG. 4 illustrates the position of the fixation device in a desired orientation relative to the leaflets.

FIGS. 5, 6A-B and 7 illustrate an embodiment of a fixation device in various positions.

FIGS. 8A-8B illustrate an embodiment of the fixation device wherein some or all of the components are molded as one part.

FIG. 9 illustrates another embodiment of the fixation device of the present disclosure.

FIGS. 10A-10B, 11A-11B, 12A-12B, 13A-13B, 14-16 illustrate embodiments of a fixation device in various possible positions during introduction and placement of the device within the body to perform a therapeutic procedure.

FIGS. 17A-17C illustrate a covering on the fixation device wherein the device is in various positions.

FIG. 18 is a schematic representation showing actuation of a stiffening rod.

FIGS. 19A-B are schematic representations showing actuation of a stiffening rod within an interventional tool.

FIGS. 20A-B are schematic representations of several examples of an inner conduit.

FIGS. 21A-B are schematic representations showing actuation of an intermediate support within an outer sheath of an interventional tool.

FIGS. 22A-B are schematic representations showing actuation of an intermediate support within a steerable sleeve of an interventional tool.

FIGS. 22C-E are schematic representations showing examples of flared ends of an intermediate support.

FIG. 23 is a schematic representation of a handle of an interventional tool having a knob for actuating the intermediate support.

FIGS. 24A-E are schematic representations showing components of an interventional tool having a support sheath.

FIG. 24F is a schematic representation of a handle of an interventional tool having a lever for actuating the support sheath.

### DETAILED DESCRIPTION

When used in connection with a delivery device for transporting a device into a patient, the terms “proximal” and “distal” are to be taken as relative to the user of the delivery devices. “Proximal” is to be understood as relatively close to the user, and “distal” is to be understood as relatively farther away from the user. When used in connection with a fixation device, the terms “proximal” and “distal” are to be taken as relative to the site of treatment. “Proximal” is to be understood as relatively close to the treatment site, and “distal” is to be understood as relatively farther away from the treatment site. As used herein, the terms “substantially,” “generally,” “approximately,” and “about” are intended to mean that slight deviations from absolute are included within the scope of the term so modified. Throughout the disclosure, the mitral valve is described in an illustrative manner. Clips may be similarly used to treat the tricuspid valve to reduce regurgitation in the right side of the heart. This tricuspid valve repair approach is particularly hindered by poor imaging due to the unfavorable anatomy of the heart in relation to the esophagus. A trans-esophageal echocardiography probe can be pressed favorably toward the left side of the heart to obtain adequate imaging of the mitral valve. This is not the case for the tricuspid valve, so imaging is generally poorer. For this reason, a sensor may provide a special benefit for users to gain confidence in implanting clips in tricuspid repair procedures. Thus, the disclosure is not limited to mitral valve clips, but similar techniques may also be used to ensure proper attachment of other clips, valves or other devices in cardiac and other medical applications.

### I. Cardiac Physiology

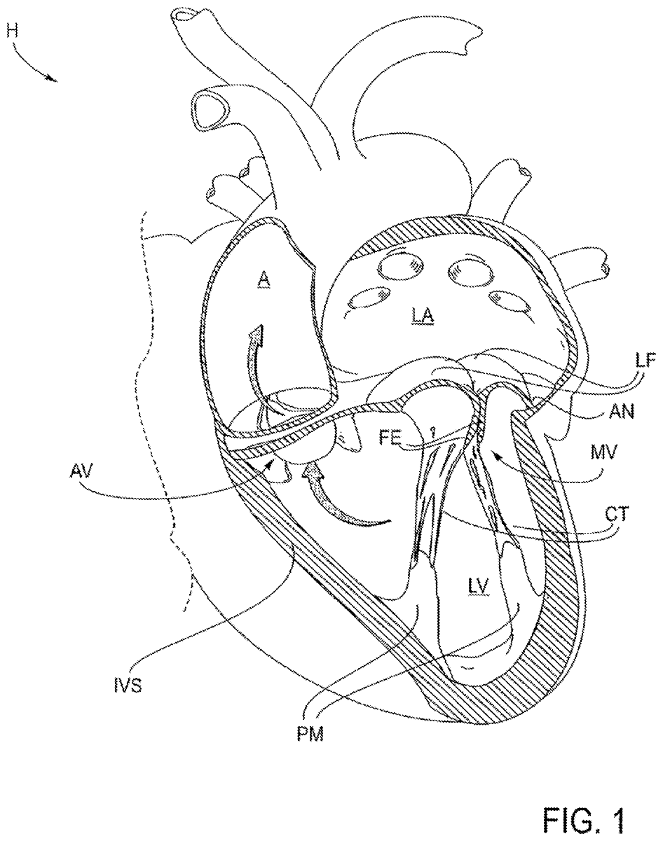

The left ventricle LV of a normal heart H in systole is illustrated in FIG. 1. The left ventricle LV is contracting and blood flows outwardly through the aortic valve AV in the direction of the arrows. Back flow of blood or “regurgitation” through the mitral valve MV is prevented since the mitral valve is configured as a “check valve” which prevents back flow when pressure in the left ventricle is higher than that in the left atrium LA. The mitral valve MV comprises a pair of leaflets having free edges FE which meet evenly to close, as illustrated in FIG. 1. The opposite ends of the leaflets LF are attached to the surrounding heart structure along an annular region referred to as the annulus AN. The free edges FE of the leaflets LF are secured to the lower portions of the left ventricle LV through chordac tendinac CT (referred to hereinafter as the chordac) which include plurality of branching tendons secured over the lower surfaces of each of the valve leaflets LF. The chordac CT in turn, are attached to the papillary muscles PM which extend upwardly from the lower portions of the left ventricle and intraventricular septum IVS.

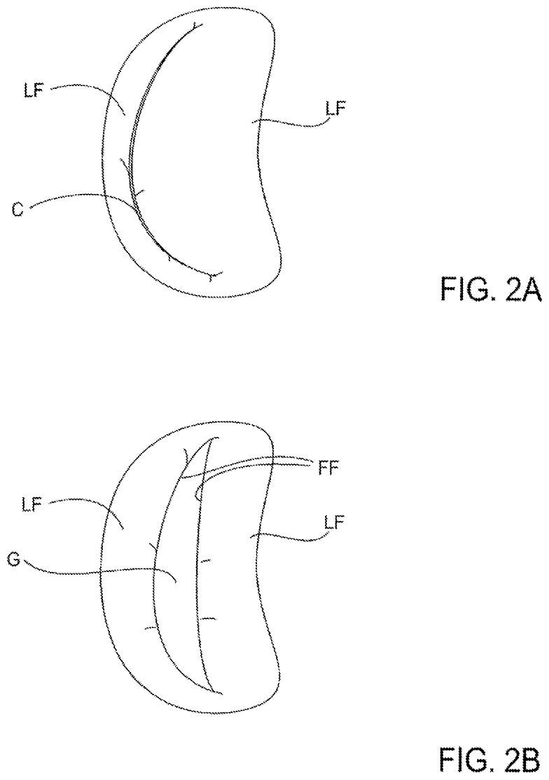

A number of structural defects in the heart can cause mitral valve regurgitation. Regurgitation occurs when the valve leaflets do not close properly allowing leakage from the ventricle into the atrium. As shown in FIG. 2A, the free edges of the anterior and posterior leaflets normally meet along a line of coaptation C. An example of a defect causing regurgitation is shown in FIG. 2B. Here an enlargement of the heart causes the mitral annulus to become enlarged, making it impossible for the free edges FE to meet during systole. This results in a gap G which allows blood to leak through the valve during ventricular systole. Ruptured or elongated chordac can also cause a valve leaflet to prolapse since inadequate tension is transmitted to the leaflet via the chordac. While the other leaflet maintains a normal profile, the two valve leaflets do not properly meet and leakage from the left ventricle into the left atrium will occur. Such regurgitation can also occur in patients who have suffered ischemic heart disease where the left ventricle does not contract sufficiently to effect proper closure.

### II. General Overview

The present disclosure provides methods and devices for grasping, approximating and fixating tissues such as valve leaflets to treat cardiac valve regurgitation, particularly mitral valve regurgitation. The present disclosure also provides features that allow repositioning and removal of the device if so desired, particularly in areas where removal may be hindered by anatomical features such as chordac CT. Such removal would allow the surgeon to reapproach the valve in a new manner if so desired.

Grasping will preferably be atraumatic providing a number of benefits. By atraumatic, it is meant that the devices and methods of the disclosure may be applied to the valve leaflets and then removed without causing any significant clinical impairment of leaflet structure or function. The leaflets and valve continue to function substantially the same as before the disclosure was applied. Thus, some minor penetration or denting of the leaflets may occur using the disclosure while still meeting the definition of “atraumatic”. This enables the devices of the disclosure to be applied to a diseased valve and, if desired, removed or repositioned without having negatively affected valve function. In addition, it will be understood that in some cases it may be necessary or desirable to pierce or otherwise permanently affect the leaflets during either grasping, fixing or both. In some of these cases, grasping and fixation may be accomplished by a single device. Although a number of embodiments are provided to achieve these results, a general overview of the basic features will be presented herein. Such features are not intended to limit the scope of the disclosure and are presented with the aim of providing a basis for descriptions of individual embodiments presented later in the application.

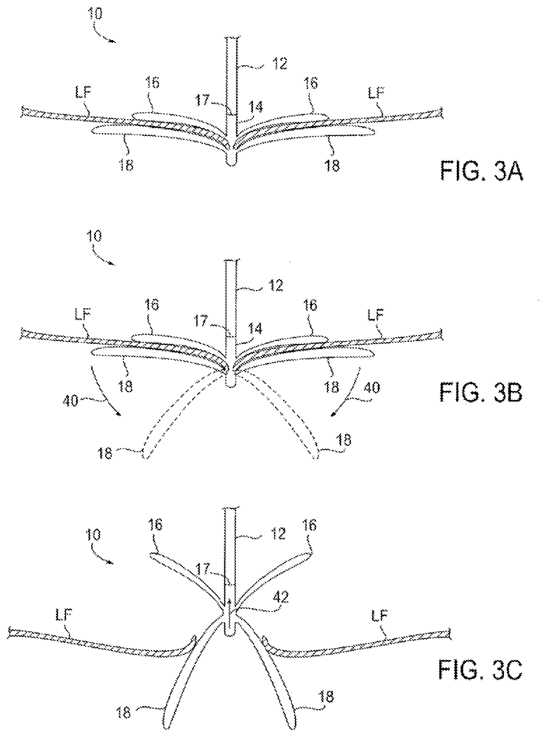

The devices and methods of the disclosure rely upon the use of an interventional tool that is positioned near a desired treatment site and used to grasp the target tissue. In endovascular applications, the interventional tool is typically an interventional catheter. In surgical applications, the interventional tool is typically an interventional instrument. In preferred embodiments, fixation of the grasped tissue is accomplished by maintaining grasping with a portion of the interventional tool which is left behind as an implant. While the disclosure may have a variety of applications for tissue approximation and fixation throughout the body, it is particularly well adapted for the repair of valves, especially cardiac valves such as the mitral valve. Referring to FIG. 3A, an interventional tool 10, having a delivery device, such as a shaft 12, and a fixation device 14, is illustrated having approached the mitral valve MV from the atrial side and grasped the leaflets LF. The mitral valve may be accessed either surgically or by using endovascular techniques, and either by a retrograde approach through the ventricle or by an antegrade approach through the atrium, as described above. For illustration purposes, an antegrade approach is described.

The fixation device 14 is releasably attached to the shaft 12 of the interventional tool 10 at its distal end. When describing the devices of the disclosure herein, “proximal” shall mean the direction toward the end of the device to be manipulated by the user outside the patient's body, and “distal” shall mean the direction toward the working end of the device that is positioned at the treatment site and away from the user. With respect to the mitral valve, proximal shall refer to the atrial or upstream side of the valve leaflets and distal shall refer to the ventricular or downstream side of the valve leaflets.

The fixation device 14 typically comprises proximal elements 16 (or gripping elements) and distal elements 18 (or fixation elements) which protrude radially outward and are positionable on opposite sides of the leaflets LF as shown so as to capture or retain the leaflets therebetween. The proximal elements 16 are preferably comprised of cobalt chromium, nitinol or stainless steel, and the distal elements 18 are preferably comprised of cobalt chromium or stainless steel, however any suitable materials may be used. The fixation device 14 is coupleable to the shaft 12 by a coupling mechanism 17. The coupling mechanism 17 allows the fixation device 14 to detach and be left behind as an implant to hold the leaflets together in the coapted position.

In some situations, it may be desired to reposition or remove the fixation device 14 after the proximal elements 16, distal elements 18, or both have been deployed to capture the leaflets LF. Such repositioning or removal may be desired for a variety of reasons, such as to reapproach the valve in an attempt to achieve better valve function, more optimal positioning of the device 14 on the leaflets, better purchase on the leaflets, to detangle the device 14 from surrounding tissue such as chordae, to exchange the device 14 with one having a different design, or to abort the fixation procedure, to name a few. To facilitate repositioning or removal of the fixation device 14 the distal elements 18 are releasable and optionally invertible to a configuration suitable for withdrawal of the device 14 from the valve without tangling or interfering with or damaging the chordac, leaflets or other tissue. FIG. 3B illustrates inversion wherein the distal elements 18 are movable in the direction of arrows 40 to an inverted position. Likewise, the proximal elements 16 may be raised, if desired. In the inverted position, the device 14 may be repositioned to a desired orientation wherein the distal elements may then be reverted to a grasping position against the leaflets as in FIG. 3A. Alternatively, the fixation device 14 may be withdrawn (indicated by arrow 42) from the leaflets as shown in FIG. 3C. Such inversion reduces trauma to the leaflets and minimizes any entanglement of the device with surrounding tissues. Once the device 14 has been withdrawn through the valve leaflets, the proximal and distal elements may be moved to a closed position or configuration suitable for removal from the body or for reinsertion through the mitral valve.

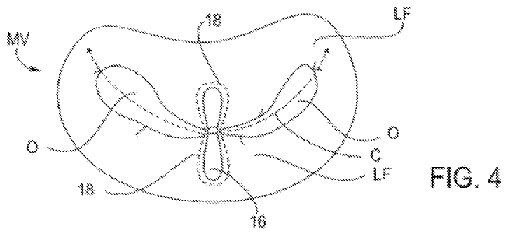

FIG. 4 illustrates the position of the fixation device 14 in a desired orientation in relation to the leaflets LF. This is a short-axis view of the mitral valve MV from the atrial side, therefore, the proximal elements 16 are shown in solid line and the distal elements 18 are shown in dashed line. The proximal and distal elements 16, 18 are positioned to be substantially perpendicular to the line of coaptation C. The device 14 may be moved roughly along the line of coaptation to the location of regurgitation. The leaflets LF are held in place so that during diastole, as shown in FIG. 4, the leaflets LF remain in position between the elements 16, 18 surrounded by openings O which result from the diastolic pressure gradient. Advantageously, leaflets LF are coapted such that their proximal or upstream surfaces are facing each other in a vertical orientation, parallel to the direction of blood flow through mitral valve MV. The upstream surfaces may be brought together so as to be in contact with one another or may be held slightly apart, but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting mitral flow pattern is satisfactory, the leaflets may be fixed together in this orientation. If the resulting color Doppler image shows insufficient improvement in mitral regurgitation, the interventional tool 10 may be repositioned. This may be repeated until an optimal result is produced wherein the leaflets LF are held in place.

Once the leaflets are coapted in the desired arrangement, the fixation device 14 is then detached from the shaft 12 and left behind as an implant to hold the leaflets together in the coapted position. As mentioned previously, the fixation device 14 is coupled to the shaft 12 by a coupling mechanism 17. Other coupling mechanisms are described in U.S. Pat. No. 9,510,829, which is hereby incorporated by reference in its entirety as if fully set forth herein.

### III. Fixation Device

### A. Introduction and Placement of Fixation Device

The fixation device 14 is delivered to the valve or the desired tissues with the use of a delivery device. The delivery device may be rigid or flexible depending on the application. For endovascular applications, the delivery device comprises a flexible delivery catheter which will be described in later sections. Typically, however, such a catheter comprises a shaft, having a proximal end and a distal end, and a fixation device releasably attached to its distal end. The shaft is usually elongate and flexible, suitable for intravascular introduction. Alternatively, the delivery device may comprise a shorter and less flexible interventional instrument which may be used for trans-thoracic surgical introduction through the wall of the heart, although some flexibility and a minimal profile will generally be desirable. A fixation device is releasably coupleable with the delivery device as illustrated in FIG. 3A. The fixation device may have a variety of forms, a few embodiments of which will be described herein.

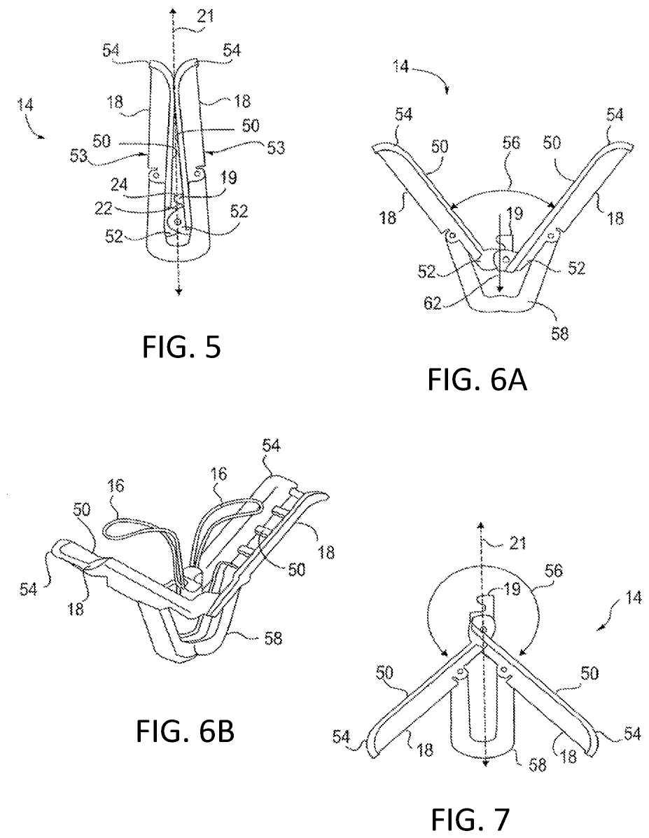

FIGS. 5, 6A-B and 7 illustrate an embodiment of a fixation device 14 in various positions or configurations. FIG. 5 illustrates the fixation device 14 in a closed configuration for delivery through the patient's vasculature and, in this example, through the mitral valve. The fixation device 14 includes a coupling member 19 which allows detachment of the fixation device 14 for implantation. In this example, the coupling member 19 is shown to include the lower shaft 22 and mating surface 24, and therefore the coupling member 19 would function similarly as described above. The fixation device 14 also includes a pair of opposed distal elements 18, each distal element 18 having an engagement surface 50 facing inwardly toward the opposed distal element 18 in the closed configuration. Distal elements 18 preferably comprise elongate arms 53, each arm having a proximal end 52 rotatably connected to the coupling member 19 and a free end 54. Suitable connections for arms 53 to coupling member 19 include pins, living hinges, or other known rotational connection mechanisms. In the closed configuration of FIG. 5, free ends 54 point in a first direction such that the arms 53 and engagement surfaces 50 are nearly parallel to each other and to an axis 21, and preferably are angled slightly inwardly toward each other. In a preferred embodiment, when tissue is not present between arms 53, the arms 53 may be closed until free ends 54 either touch each other or engage shaft 12 when fixation device 14 is attached thereto, thereby minimizing the profile of the fixation device 14 for passage through a delivery device.

FIGS. 6A-B illustrate the fixation device 14 in an open position wherein the engagement surfaces 50 are disposed at a separation angle 56 apart, wherein the separation angle 56 is typically up to approximately 180 degrees, preferably up to 90-180 degrees, and arms 53 are disposed generally symmetrically relative to axis 21. The arms 53 may be movable to the open position by a variety of actuation mechanisms. For example, a plunger or actuator rod may be advanced through the coupling member 19, as indicated by arrow 62, so as to engage a spring or spring loaded actuation mechanism 58 which is attached to the distal elements 18. By exerting a force against the actuation mechanism 58, the distal elements 18 are rotated relative to coupling member 19. The distal elements 18 may be held in this open position by the actuator rod against the resistance provided by the spring of the actuation mechanism 58 which biases the distal elements 18 toward the closed position of FIG. 5 when the distal elements 18 are less than 180 degrees apart. The spring loading of the actuation mechanism 58 resists outward movement of the actuation mechanism 58 and urges the device 14 towards the closed position.

In this embodiment, proximal elements 16 comprise resilient loop-shaped wire forms biased outwardly and attached to the coupling member 19 so as to be biased to an open position shown in FIG. 6B but movable rotationally inwardly when arms 53 are closed. The wire forms may be flexible enough to be rigidly attached to coupling member 19 and resiliently deflectable inwardly, or they may be attached by a rotational coupling such as a pin or living hinge. In use, leaflets LF are positioned between the proximal elements 16 and distal elements 18. Once, the leaflets LF are positioned between the proximal and distal elements 16, 18, the distal elements 18 may be closed, compressing the leaflets between engagement surfaces 50 and proximal elements 18. Depending upon the thickness of the leaflets, the arrangements of the leaflets, the position of the fixation device on the leaflets and other factors, the arms 53 may be maintained in the open position of FIGS. 6A-B, moved to the fully closed position of FIG. 5, or placed in any of various positions in between so as to coapt the leaflets LF and hold them in the desired position with the desired degree of force. In any case, the fixation device 14 will remain in place as an implant following detachment from the delivery catheter.

In some situations, as previously mentioned, it may be desirable to reopen the fixation device 14 following initial placement. To reopen the device 14, the actuator rod may be readvanced or reinserted through the coupling member 19 and readvanced to press against the actuation mechanism 58, as previously indicated by arrow 62 in FIG. 6A. Again, such advancement applies a force against the actuation mechanism 58 in the manner described above thus moving arms 53 outwardly to release force against leaflets and move engagement surfaces 50 away from proximal elements 16. The leaflets are then free to move relative to fixation device 14. The fixation device 14 may then be repositioned as desired and the actuator rod refracted to reclose the distal elements 18 to coapt the leaflets.

Under some circumstances, it may be further desirable to withdraw the fixation device 14 back through the valve or completely from the patient following initial insertion through the valve. Should this be attempted with the clip in the closed or open positions illustrated in FIGS. 5, 6A-B and 7, there may be a risk that arms 53 could interfere or become entangled with the chordac, leaflets or other tissues. To avoid this, the fixation element 14 is preferably adapted for inversion of arms 53 so that free ends 54 point in a second direction, opposite to the first direction in which the free ends 54 pointed in the closed position, each arm 53 forming an obtuse angle relative to axis 21 as illustrated in FIG. 7. The arms 53 may be rotated so that the engagement surfaces 50 are disposed at a separation angle 56 of up to 360 degrees, and preferably at least up to 270 degrees. This may be accomplished by exerting a force against actuation mechanism 58 with a push rod or plunger extending through coupling member 19 as described above. In this embodiment, once the distal elements 18 have rotated beyond 180 degrees apart, the spring loading of the actuation mechanism 58 biases the distal elements 18 toward the inverted position. The spring loading of the actuation mechanism 58 resists outward movement of the actuation mechanism 58 and urges the device 14 towards the inverted position.

With arms 53 in the inverted position, engagement surfaces 50 provide an atraumatic surface deflect tissues as the fixation device is withdrawn. This allows the device to be retracted back through the valve annulus without risk of injury to valvular and other tissues. In some cases, once the fixation device 14 has been pulled back through the valve, it will be desirable to return the device to the closed position for withdrawal of the device from the body (either through the vasculature or through a surgical opening).

The embodiment illustrated in FIGS. 5, 6A-B and 7 is assembled from separate components composed of biocompatible materials. The components may be formed from the same or different materials, including but not limited to stainless steel or other metals, Elgiloy®, nitinol, titanium, tantalum, metal alloys or polymers. Additionally, some or all of these components may be made of bioabsorbable materials that will be absorbed by surrounding tissues or will dissolve into the bloodstream following implantation. It has been found that in mitral valve repair applications the fixation devices of the disclosure are completely surrounded by tissue within a few months of implantation, after which the devices could dissolve or be absorbed without negative impact to the repair.

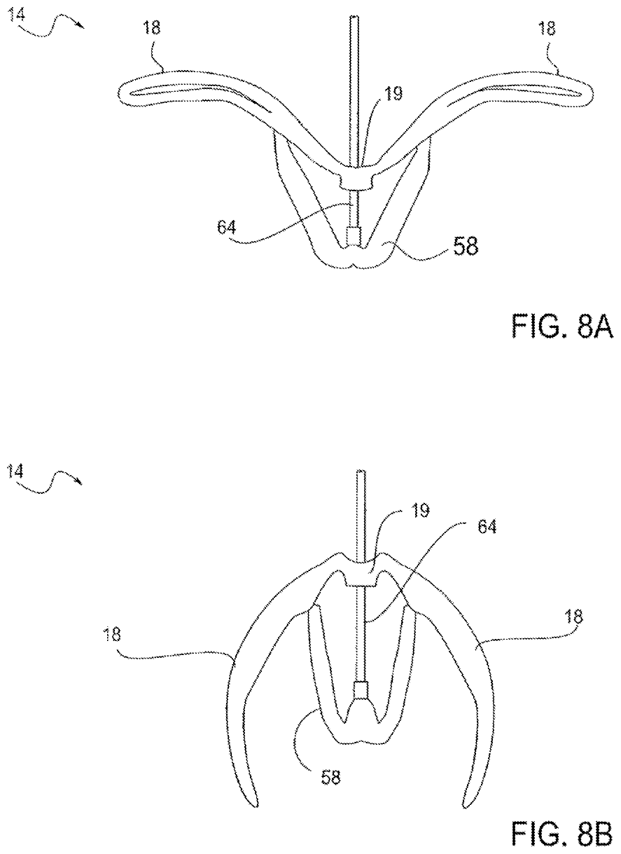

In a further embodiment, some or all of the components may be molded as one part, as illustrated in FIGS. 8A-8B. Here, the coupling member 19, distal elements 18 and actuation mechanism 58 of the fixation device 14 are all molded from a polymer material as one movable piece. FIG. 8A shows the fixation device 14 in the open position. Advancement of an actuator rod 64 rotates the distal elements 18 relative to the coupling member 19 by a living hinge or by elastic deformation of the plastic at the point of connection between the elements 18 and the coupling member 19. Typically, this point of connection comprises a thinner segment of polymer to facilitate such bending. Likewise, the actuation mechanism 58 coupled to the distal elements 18 in the same manner. FIG. 8B shows the fixation device 14 in the inverted position.

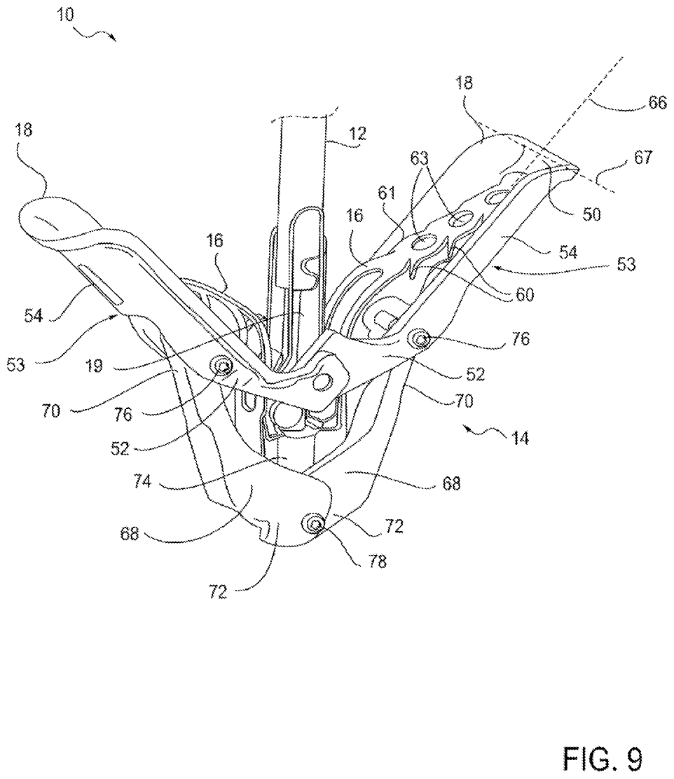

FIG. 9 illustrates another embodiment of a fixation device 14. Here, the fixation device 14 is shown coupled to a shaft 12 to form an interventional tool 10. The fixation device 14 includes a coupling member 19 and a pair of opposed distal elements 18. The distal elements 18 comprise elongate arms 53, each arm having a proximal end 52 rotatably connected to the coupling member 19 and a free end 54. The free ends 54 have a rounded shape to minimize interference with and trauma to surrounding tissue structures. Preferably, each free end 54 defines a curvature about two axes, one being an axis 66 perpendicular to longitudinal axis of arms 53. Thus, the engagement surfaces 50 have a cupped or concave shape to surface area in contact with tissue and to assist in grasping and holding the valve leaflets. This further allows arms 53 to nest around the shaft 12 in the closed position to minimize the profile of the device. Preferably, arms 53 are at least partially cupped or curved inwardly about their longitudinal axes 66. Also, preferably, each frec end 54 defines a curvature about an axis 67 perpendicular to axis 66 or the longitudinal axis of arms 53. This curvature is a reverse curvature along the most distal portion of the free end 54. Likewise, the longitudinal edges of the free ends 54 may flare outwardly. Both the reverse curvature and flaring minimize trauma to the tissue engaged therewith.

In a preferred embodiment suitable for mitral valve repair, the transverse width across engagement surfaces 50 (which determines the width of tissue engaged) is at least about 2 mm, usually 3-10 mm, and preferably about 4-6 mm. In some situations, a wider engagement is desired wherein the engagement surfaces 50 are larger, for example about 2 cm, or multiple fixation devices are used adjacent to each other. Arms 53 and engagement surfaces 50 are configured to engage a length of tissue of about 4-10 mm, and preferably about 6-8 mm along the longitudinal axis of arms 53. Arms 53 further include a plurality of openings to enhance grip and to promote tissue ingrowth following implantation.

The valve leaflets are grasped between the distal elements 18 and proximal elements 16. In some embodiments, the proximal elements 16 are flexible, resilient, and cantilevered from coupling member 19. The proximal elements are preferably resiliently biased toward the distal elements. Each proximal element 16 is shaped and positioned to be at least partially recessed within the concavity of the distal element 18 when no tissue is present. When the fixation device 14 is in the open position, the proximal elements 16 are shaped such that each proximal element 16 is separated from the engagement surface 50 near the proximal end 52 of arm 53 and slopes toward the engagement surface 50 near the free end 54 with the free end of the proximal element contacting engagement surface 50, as illustrated in FIG. 9. This shape of the proximal elements 16 accommodates valve leaflets or other tissues of varying thicknesses.

Proximal elements 16 include a plurality of openings 63 and scalloped side edges 61 to increase grip on tissue. The proximal elements 16 optionally include frictional accessories, frictional features or grip-enhancing elements to assist in grasping and/or holding the leaflets. In preferred embodiments, the frictional accessories comprise barbs 60 having tapering pointed tips extending toward engagement surfaces 50. It may be appreciated that any suitable frictional accessories may be used, such as prongs, windings, bands, barbs, grooves, channels, bumps, surface roughening, sintering, high-friction pads, coverings, coatings or a combination of these.

Optionally, magnets may be present in the proximal and/or distal elements. It may be appreciated that the mating surfaces will be made from or will include material of opposite magnetic charge to cause attraction by magnetic force. For example, the proximal elements and distal elements may each include magnetic material of opposite charge so that tissue is held under constant compression between the proximal and distal elements to facilitate faster healing and ingrowth of tissue. Also, the magnetic force may be used to draw the proximal elements 16 toward the distal elements 18, in addition to or alternatively to biasing of the proximal elements toward the distal elements. This may assist in deployment of the proximal elements 16. In another example, the distal elements 18 each include magnetic material of opposite charge so that tissue positioned between the distal elements 18 is held therebetween by magnetic force.

The proximal elements 16 may be covered with a fabric or other flexible material as described below to enhance grip and tissue ingrowth following implantation. Preferably, when fabrics or coverings are used in combination with barbs or other frictional features, such features will protrude through such fabric or other covering so as to contact any tissue engaged by proximal elements 16.

In an exemplary embodiment, proximal elements 16 are formed from metallic sheet of a spring-like material using a stamping operation which creates openings 63, scalloped edges 61 and barbs 60. Alternatively, proximal elements 16 could be comprised of a spring-like material or molded from a biocompatible polymer. It should be noted that while some types of frictional accessories that can be used in the present disclosure may permanently alter or cause some trauma to the tissue engaged thereby, in a preferred embodiment, the frictional accessories will be atraumatic and will not injure or otherwise affect the tissue in a clinically significant way. For example, in the case of barbs 60, it has been demonstrated that following engagement of mitral valve leaflets by fixation device 14, should the device later be removed during the procedure barbs 60 leave no significant permanent scarring or other impairment of the leaflet tissue and are thus considered atraumatic.

The fixation device 14 also includes an actuation mechanism 58. In this embodiment, the actuation mechanism 58 comprises two link members or legs 68, each leg 68 having a first end 70 which is rotatably joined with one of the distal elements 18 at a riveted joint 76 and a second end 72 which is rotatably joined with a stud 74. The legs 68 are preferably comprised of a rigid or semi-rigid metal or polymer such as Elgiloy®, cobalt chromium or stainless steel, however any suitable material may be used. While in the embodiment illustrated both legs 68 are pinned to stud 74 by a single rivet 78, it may be appreciated, however, that each leg 68 may be individually attached to the stud 74 by a separate rivet or pin. The stud 74 is joinable with an actuator rod 64 (not shown) which extends through the shaft 12 and is axially extendable and retractable to move the stud 74 and therefore the legs 68 which rotate the distal elements 18 between closed, open and inverted positions. Likewise, immobilization of the stud 74 holds the legs 68 in place and therefore holds the distal elements 18 in a desired position. The stud 74 may also be locked in place by a locking feature which will be further described in later sections.

In any of the embodiments of fixation device 14 disclosed herein, it may be desirable to provide some mobility or flexibility in distal elements 18 and/or proximal elements 16 in the closed position to enable these elements to move or flex with the opening or closing of the valve leaflets. This provides shock absorption and thereby reduces force on the leaflets and minimizes the possibility for tearing or other trauma to the leaflets. Such mobility or flexibility may be provided by using a flexible, resilient metal or polymer of appropriate thickness to construct the distal elements 18. Also, the locking mechanism of the fixation device (described below) may be constructed of flexible materials to allow some slight movement of the proximal and distal elements even when locked. Further, the distal elements 18 can be connected to the coupling mechanism 19 or to actuation mechanism 58 by a mechanism that biases the distal element into the closed position (inwardly) but permits the arms to open slightly in response to forces exerted by the leaflets. For example, rather than being pinned at a single point, these components may be pinned through a slot that allowed a small amount of translation of the pin in response to forces against the arms. A spring is used to bias the pinned component toward one end of the slot.

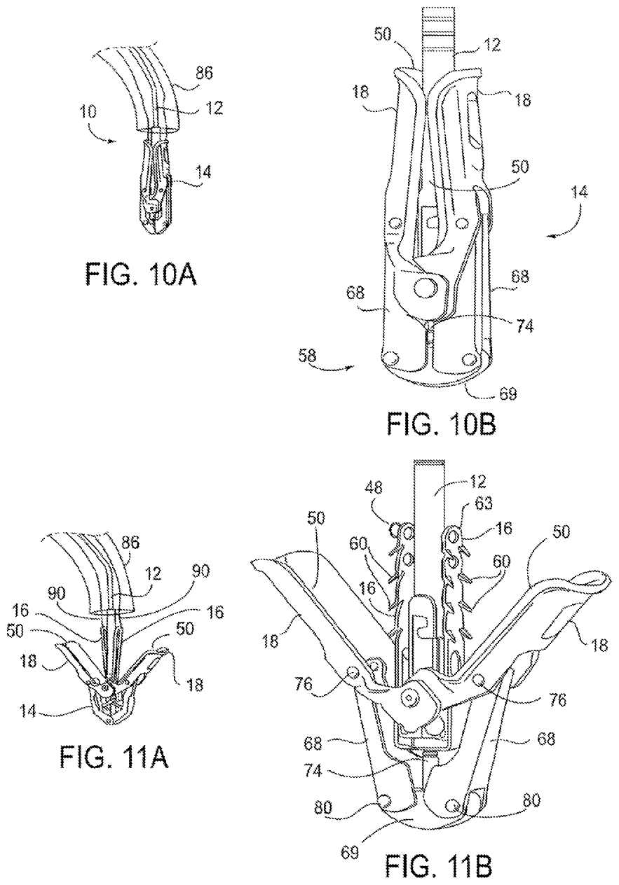

FIGS. 10A-10B, 11A-11B, 12A-12B, 13A-13B, and FIGS. 14-16 illustrate embodiments of the fixation device 14 of FIG. 9 in various possible positions during introduction and placement of the device 14 within the body to perform a therapeutic procedure. FIG. 10A illustrates an embodiment of an interventional tool 10 delivered through an inner conduit 86. It may be appreciated that the interventional tool 10 may take the form of a catheter, and likewise, the inner conduit 86 may take the form of a guide catheter or sheath. However, in this example the terms interventional tool 10 and inner conduit 86 will be used. The interventional tool 10 comprises a fixation device 14 coupled to a shaft 12 and the fixation device 14 is shown in the closed position. FIG. 10B illustrates a similar embodiment of the fixation device of FIG. 10A in a larger view. In the closed position, the opposed pair of distal elements 18 are positioned so that the engagement surfaces 50 face each other. Each distal element 18 comprises an elongate arm 53 having a cupped or concave shape so that together the arms 53 surround the shaft 12 and optionally contact each other on opposite sides of the shaft. This provides a low profile for the fixation device 14 which is readily passable through the inner conduit 86 and through any anatomical structures, such as the mitral valve. In addition, FIG. 10B further includes an actuation mechanism 58. In this embodiment, the actuation mechanism 58 comprises two legs 68 which are each movably coupled to a base 69. The base 69 is joined with an actuator rod 64 which extends through the shaft 12 and is used to manipulate the fixation device 14. In some embodiments, the actuator rod 64 attaches directly to the actuation mechanism 58, particularly the base 69. However, the actuator rod 64 may alternatively attach to a stud 74 which in turn is attached to the base 69. In some embodiments, the stud 74 is threaded so that the actuator rod 64 attaches to the stud 74 by a screw-type action. However, the rod 64 and stud 74 may be joined by any mechanism which is releasable to allow the fixation device 14 to be detached from shaft 12.

FIGS. 11A-11B illustrate the fixation device 14 in the open position. In the open position, the distal elements 18 are rotated so that the engagement surfaces 50 face a first direction. Distal advancement of the stud 74 relative to coupling member 19 by action of the actuator rod 64 applies force to the distal elements 18 which begin to rotate around joints 76 due to freedom of movement in this direction. Such rotation and movement of the distal elements 18 radially outward causes rotation of the legs 68 about joints 80 so that the legs 68 are directly slightly outwards. The stud 74 may be advanced to any desired distance correlating to a desired separation of the distal elements 18. In the open position, engagement surfaces 50 are disposed at an acute angle relative to shaft 12, and are preferably at an angle of between 90 and 180 degrees relative to each other. In one embodiment, in the open position the free ends 54 of arms 53 have a span therebetween of about 10-20 mm, usually about 12-18 mm, and preferably about 14-16 mm.

Proximal elements 16 are typically biased outwardly toward arms 53. The proximal elements 16 may be moved inwardly toward the shaft 12 and held against the shaft 12 with the aid of proximal element lines 90 which can be in the form of sutures, wires, nitinol wire, rods, cables, polymeric lines, or other suitable structures. The proximal element lines 90 may be connected with the proximal elements 16 by threading the lines 90 in a variety of ways. When the proximal elements 16 have a loop shape, as shown in FIG. 11A, the line 90 may pass through the loop and double back. When the proximal elements 16 have an elongate solid shape, as shown in FIG. 11B, the line 90 may pass through one or more of the openings 63 in the element 16. Further, a line loop 48 may be present on a proximal element 16, also illustrated in FIG. 11B, through which a proximal element line 90 may pass and double back. Such a line loop 48 may be useful to reduce friction on proximal element line 90 or when the proximal elements 16 are solid or devoid of other loops or openings through which the proximal element lines 90 may attach. A proximal element line 90 may attach to the proximal elements 16 by detachable means which would allow a single line 90 to be attached to a proximal element 16 without doubling back and would allow the single line 90 to be detached directly from the proximal element 16 when desired. Examples of such detachable means include hooks, snares, clips or breakable couplings, to name a few. By applying sufficient tension to the proximal element line 90, the detachable means may be detached from the proximal element 16 such as by breakage of the coupling. Other mechanisms for detachment may also be used. Similarly, a lock line 92 may be attached and detached from a locking mechanism by similar detachable means.

In the open position, the fixation device 14 can engage the tissue which is to be approximated or treated. The embodiment illustrated in FIGS. 9-11 is adapted for repair of the mitral valve using an antegrade approach from the left atrium. The interventional tool 10 is advanced through the mitral valve from the left atrium to the left ventricle. The distal elements 18 are oriented to be perpendicular to the line of coaptation and then positioned so that the engagement surfaces 50 contact the ventricular surface of the valve leaflets, thereby grasping the leaflets. The proximal elements 16 remain on the atrial side of the valve leaflets so that the leaflets lie between the proximal and distal elements. In this embodiment, the proximal elements 16 have frictional accessories, such as barbs 60 which are directed toward the distal elements 18. However, neither the proximal elements 16 nor the barbs 60 contact the leaflets at this time.

The interventional tool 10 may be repeatedly manipulated to reposition the fixation device 14 so that the leaflets are properly contacted or grasped at a desired location. Repositioning is achieved with the fixation device in the open position. In some instances, regurgitation may also be checked while the device 14 is in the open position. If regurgitation is not satisfactorily reduced, the device may be repositioned and regurgitation checked again until the desired results are achieved.

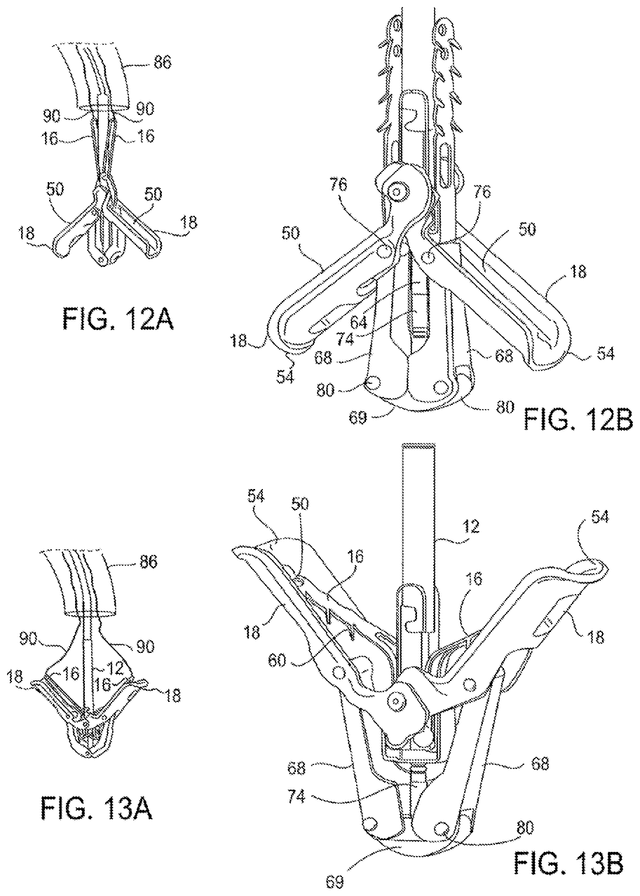

It may also be desired to invert the fixation device 14 to aid in repositioning or removal of the fixation device 14. FIGS. 12A-12B illustrate the fixation device 14 in the inverted position. By further advancement of stud 74 relative to coupling member 19, the distal elements 18 are further rotated so that the engagement surfaces 50 face outwardly and free ends 54 point distally, with each arm 53 forming an obtuse angle relative to shaft 12. The angle between arms 53 is preferably in the range of about 270 to 360 degrees. Further advancement of the stud 74 further rotates the distal elements 18 around joints 76. This rotation and movement of the distal elements 18 radially outward causes rotation of the legs 68 about joints 80 so that the legs 68 are returned toward their initial position, generally parallel to each other. The stud 74 may be advanced to any desired distance correlating to a desired inversion of the distal elements 18. Preferably, in the fully inverted position, the span between free ends 54 is no more than about 20 mm, usually less than about 16 mm, and preferably about 12-14 mm. In this illustration, the proximal elements 16 remain positioned against the shaft 12 by exerting tension on the proximal element lines 90. Thus, a relatively large space may be created between the elements 16, 18 for repositioning. In addition, the inverted position allows withdrawal of the fixation device 14 through the valve while minimizing trauma to the leaflets. Engagement surfaces 50 provide an atraumatic surface for deflecting tissue as the fixation device is refracted proximally. It should be further noted that barbs 60 are angled slightly in the distal direction (away from the free ends of the proximal elements 16), reducing the risk that the barbs will catch on or lacerate tissue as the fixation device is withdrawn.

Once the fixation device 14 has been positioned in a desired location against the valve leaflets, the leaflets may then be captured between the proximal elements 16 and the distal elements 18. FIGS. 13A-13B illustrate the fixation device 14 in such a position. Here, the proximal elements 16 are lowered toward the engagement surfaces 50 so that the leaflets are held therebetween. In FIG. 13B, the proximal elements 16 are shown to include barbs 60 which may be used to provide atraumatic gripping of the leaflets. Alternatively, larger, more sharply pointed barbs or other penetration structures may be used to pierce the leaflets to more actively assist in holding them in place. This position is similar to the open position of FIGS. 11A-11B, however the proximal elements 16 are now lowered toward arms 53 by releasing tension on proximal element lines 90 to compress the leaflet tissue therebetween. At any time, the proximal elements 16 may be raised and the distal elements 18 adjusted or inverted to reposition the fixation device 14, if regurgitation is not sufficiently reduced.

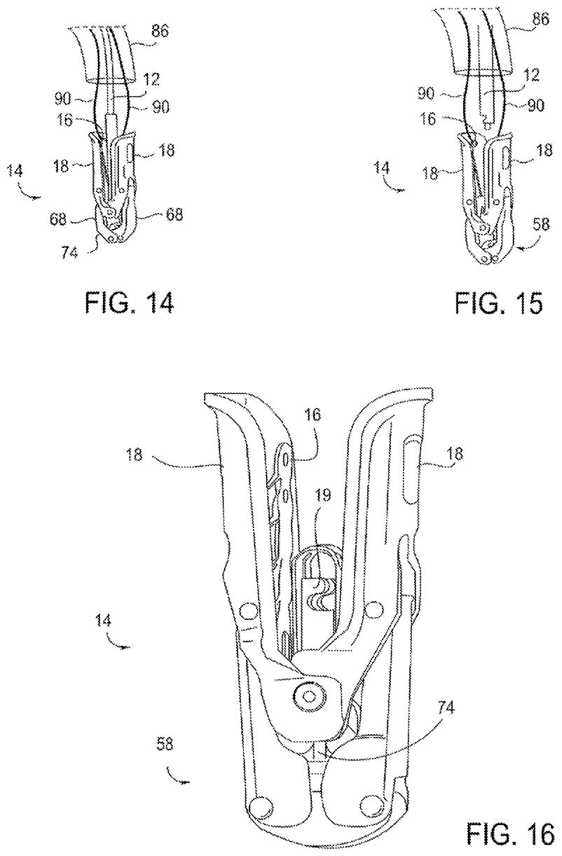

After the leaflets have been captured between the proximal and distal elements 16, 18 in a desired arrangement, the distal elements 18 may be locked to hold the leaflets in this position or the fixation device 14 may be returned to or toward a closed position. Such locking will be described in a later section. FIG. 14 illustrates the fixation device 14 in the closed position wherein the leaflets (not shown) are captured and coapted. This is achieved by retraction of the stud 74 proximally relative to coupling member 19 so that the legs 68 of the actuation mechanism 58 apply an upwards force to the distal elements 18 which in turn rotate the distal elements 18 so that the engagement surfaces 50 again face one another. The released proximal elements 16 which are biased outwardly toward distal elements 18 are concurrently urged inwardly by the distal elements 18. The fixation device 14 may then be locked to hold the leaflets in this closed position as described below.

As shown in FIG. 15, the fixation device 14 may then be released from the shaft 12. As mentioned, the fixation device 14 is releasably coupleable to the shaft 12 by coupling member 19. FIG. 15 illustrates the coupling structure, a portion of the shaft 12 to which the coupling member 19 of the fixation device 14 attaches. As shown, the proximal element lines 90 may remain attached to the proximal elements 16 following detachment from shaft 12 to function as a tether to keep the fixation device 14 connected with the catheter 86. Optionally, a separate tether coupled between shaft 12 and fixation device 14 may be used expressly for this purpose while the proximal element lines 90 are removed. In any case, the repair of the leaflets or tissue may be observed by non-invasive visualization techniques, such as echocardiography, to ensure the desired outcome. If the repair is not desired, the fixation device 14 may be retrieved with the use of the tether or proximal element lines 90 so as to reconnect coupling member 19 with shaft 12.

In an exemplary embodiment, proximal element lines 90 are elongated flexible threads, wire, cable, sutures or lines extending through shaft 12, looped through proximal elements 16, and extending back through shaft 12 to its proximal end. When detachment is desired, one end of each line may be released at the proximal end of the shaft 12 and the other end pulled to draw the free end of the line distally through shaft 12 and through proximal element 16 thereby releasing the fixation device.

FIG. 16 illustrates a released fixation device 14 in a closed position. As shown, the coupling member 19 remains separated from the shaft 12 of the interventional tool 10 and the proximal elements 16 are deployed so that tissue (not shown) may reside between the proximal elements 16 and distal elements 18.

While the above-described embodiments of the disclosure utilize a push-to-open, pull-to-close mechanism for opening and closing distal elements 18, it should be understood that a pull-to-open, push-to-close mechanism is equally possible. For example, distal elements 18 may be coupled at their proximal ends to stud 74 rather than to coupling member 19, and legs 68 may be coupled at their proximal ends to coupling member 19 rather than to stud 74. In this example, when stud 74 is pushed distally relative to coupling member 19, distal elements 18 would close, while pulling on stud 74 proximally toward coupling member 19 would open distal elements 18. Additionally, embodiments that include a spring-force closure mechanism for opening and/or closing are also contemplated.

### B. Covering on Fixation Device

The fixation device 14 may optionally include a covering. The covering may assist in grasping the tissue and may later provide a surface for tissue ingrowth. Ingrowth of the surrounding tissues, such as the valve leaflets, provides stability to the device 14 as it is further anchored in place and may cover the device with native tissue thus reducing the possibility of immunologic reactions. The covering may be comprised of any biocompatible material, such as polyethylene terephthalate, polyester, cotton, polyurethane, expanded polytetrafluoroethylene (cPTFE), silicon, or various polymers or fibers and have any suitable form, such as a fabric, mesh, textured weave, felt, looped or porous structure. Generally, the covering has a low profile so as not to interfere with delivery through an introducer sheath or with grasping and coapting of leaflets or tissue.

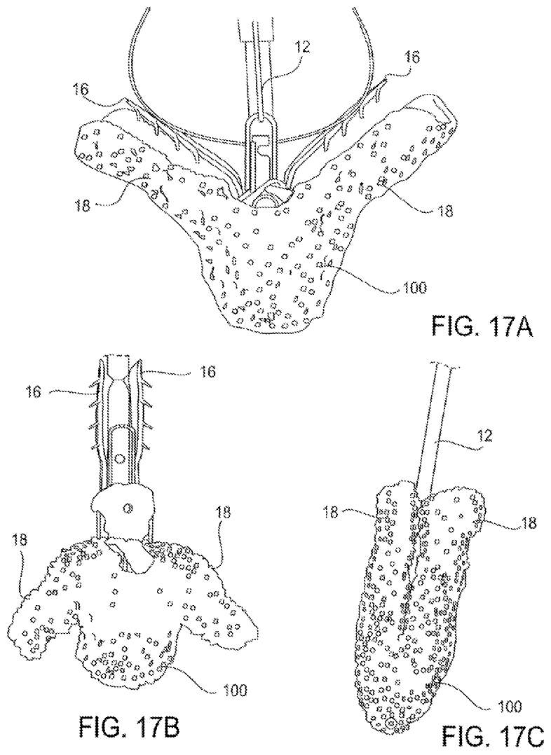

FIGS. 17A-17C illustrate a covering 100 on the fixation device 14 wherein the device 14 is in various positions. FIG. 17A shows the covering 100 encapsulating the distal elements 18 and the actuation mechanism 58 while the device 14 is in the open position. Thus, the engagement surfaces 50 are covered by the covering 100 which helps to minimize trauma on tissues and provides additional friction to assist in grasping and retaining tissues. FIG. 17B shows the device 14 of FIG. 17A in the inverted position. The covering 100 is loosely fitted and/or is flexible or elastic such that the device 14 can freely move to various positions and the covering 100 conforms to the contours of the device 14 and remains securely attached in all positions. FIG. 17C shows the device 14 in the closed position. Thus, when the fixation device 14 is left behind as an implant in the closed position, the exposed surfaces of the device 14 are substantially covered by the covering 100. It may be appreciated that the covering 100 may cover specific parts of the fixation device 14 while leaving other parts exposed. For example, the covering 100 may comprise sleeves that fit over the distal elements 18 and not the actuation mechanism 58, caps that fit over the distal ends 54 of the distal elements 18 or pads that cover the engagement surfaces 50, to name a few. It may be appreciated that, the covering 100 may allow any frictional accessories, such as barbs, to be exposed. Also, the covering 100 may cover the proximal elements 16 and/or any other surfaces of the fixation device 14. In any case, the covering 100 should be durable to withstand multiple introduction cycles and, when implanted within a heart, a lifetime of cardiac cycles.

The covering 100 may alternatively be comprised of a polymer or other suitable materials dipped, sprayed, coated or otherwise adhered to the surfaces of the fixation device 14. Optionally, the polymer coating may include pores or contours to assist in grasping the tissue and/or to promote tissue ingrowth.

Any of the coverings 100 may optionally include drugs, antibiotics, anti-thrombosis agents, or anti-platelet agents such as heparin, COUMADIN® (Warfarin Sodium), to name a few. These agents may, for example, be impregnated in or coated on the coverings 100. These agents may then be delivered to the grasped tissues surrounding tissues and/or bloodstream for therapeutic effects.

### C. Stiffening Rods for Variable Stiffness Delivery System

The disclosure above describes several variations of fixation devices and corresponding delivery devices for implanting the fixation devices within the native anatomy. When delivering an edge-to-edge valve repair implant to the mitral or tricuspid valve, a relatively stiff delivery system shaft is traditionally desired as a system with higher stiffness and stability enables accurate steering, positioning, and deployment of a fixation device or implant. While a higher stiffness may enable a delivery system to be more responsive and predictable, the delivery system catheter shaft cannot be too stiff as the system needs to flexibly bend and pass through curved venous anatomy to reach the valve. It is therefore desirable to have a delivery system that is as stiff as possible to resist leaflet motion and forces (e.g., for precise delivery to the target lesion) while still being flexible enough to navigate venous curvature. Additionally, a high degree of bending stiffness generally correlates with better torque response (as torsional stiffness is related to bending stiffness), which is particularly useful for a transcatheter edge-to-edge repair device as it may be desirable to rotate the implant so that the implant arms are perpendicular to the line of leaflet coaptation.

While high delivery system stiffness and high stability are important characteristics for repair device navigation and delivery, there is a non-intuitive drawback for such devices in that a stiff delivery catheter may enable the user to inadvertently distort a valve being grasped and repaired. Specifically, the temporary stiffness and support of a high-stiffness catheter makes assessing mitral regurgitation (MR) or tricuspid regurgitation (TR) inaccurate as there can be a large difference in the state of the repaired valve during the repair (e.g., with leaflets/valve anatomy stabilized by the catheter) and after release of the edge-to-edge repair device from the catheter. For example, subtle movements (e.g., handle rotation, advancement/retraction of the delivery catheter, etc.) may apply torsional and/or tensile/compressive loads to the leaflets and valve, of which the interventionalist may be unaware. These loads may, in turn, impact the observable mitral regurgitation or tricuspid regurgitation until the implant is deployed. Upon deployment, when the implant is no longer attached to the delivery catheter, the performance of the implant of fixation device may change. Therefore, a delivery system with variable stiffness and stability is proposed that provides (1) relatively high stiffness during device positioning, and (2) reduced stiffness prior to full device deployment and/or release (e.g., during operational testing of the implant). It may also be desirable to have a delivery system that allows a user to toggle between high-stiffness and low-stiffness catheter modes is in order to avoid applying unintentionally high forces when maneuvering and interacting with anatomy. For example, when a user grasps one leaflet independently, then translates a fixation device across a leaflet gap to the other leaflet for a second independent grasp, the user may overshoot and pull excessively on the first leaflet, causing injury, especially with quick or abrupt motion. In this two-stage grasping scenario, switching to a low-stiffness mode before moving the fixation device across the gap between leaflets may mitigate any chance of damage to the initially grasped leaflet that is getting pulled by the fixation device. The present disclosure provides several mechanisms to achieve variable stiffness.

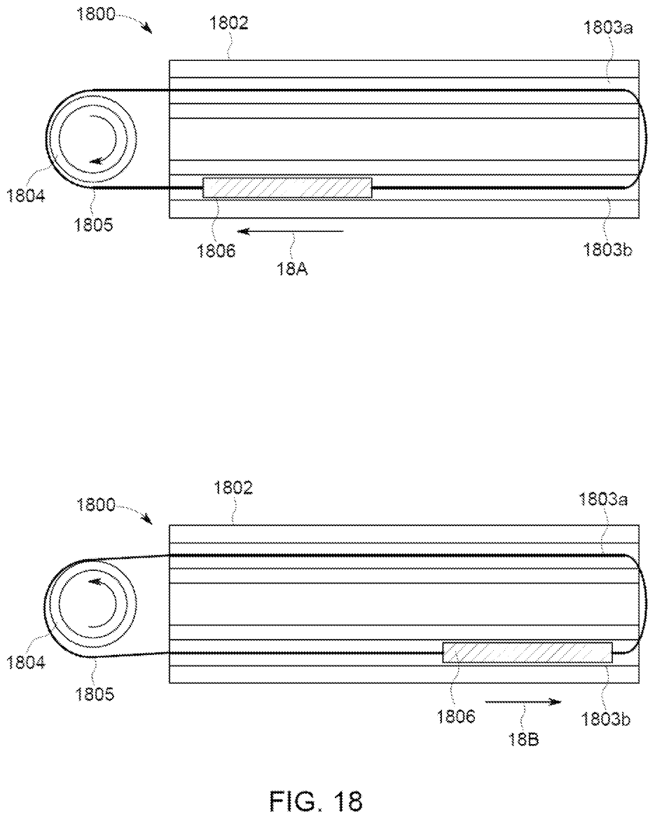

In one embodiment, variable stiffness may be achieved through one or more retractable stiffening rod(s) to adjust the stiffness of any delivery device component including an outer sheath, a steerable sleeve and an inner conduit, and any combinations thereof. FIG. 18 illustrates one potential mechanism for adjusting the stiffness of a catheter 1800. In this example, catheter 1800 may include a body 1802 defining two longitudinally-extending lumens 1803a, 1803b, a knob 1804 supporting a cable 1805 that extends through lumens 1803a, 1803b. A stiffening rod 1806 may be coupled to cable 1805 and moveable therewith through lumens 1803a, 1803b. As shown in the upper section of the drawing, rotating knob 1804 in the clockwise direction may draw the lower portion of the cable (and with it the stiffening rod 1806) proximally in the direction of arrow “18A”. Conversely, rotating knob 1804 in the counterclockwise direction may advance the lower portion of the cable (and with it the stiffening rod 1806) distally in the direction of arrow “18B” so that the absolute position of the stiffening rod 1806 with respect to the body 1802 is adjustable. In this way, a catheter 1800 having multi-lumen extrusions housing cables attached to stiffening rods may be used. Two minor lumens in the catheter shaft (or within the wall thickness of an extruded tube) may be defined for each stiffening rod 1806, but it will be appreciated that variations are possible including additional or fewer stiffening rods, and additional or fewer lumens. Thus, a stiffening rod 1806 may be retracted by the user from a distal section to a proximal section to make the distal catheter more flexible. Alternatively, a stiffening rod 1806 may be advanced into a distal section of the catheter to increase its stiffness by rotating a pulley knob on the handle.

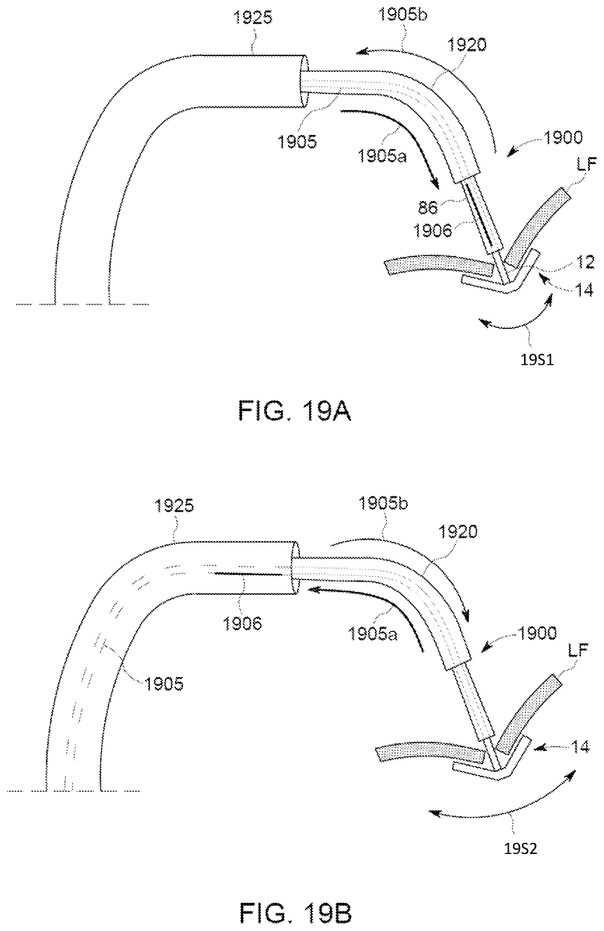

FIGS. 19A-B illustrate a delivery device 1900 with a stiffening rod 1906 moveable between two positions to transition the delivery device between a rigid state 19S1 (FIG. 19A) and a flexible state 19S2 (FIG. 19B). In FIG. 19A, a delivery device 1900 having a shaft 12, an inner conduit 86, a steerable sleeve 1920, and an outer sheath 1925 is shown, the delivery device 1900 being coupled to a fixation device 14. In the rigid state 19S1 shown in FIG. 19A, stiffening rod 1906 is disposed within inner conduit 86 at a relatively distal position, with additional stiffness presented at the distal end of the inner conduit 86 for more accurate steering, positioning and/or movement. A knob or other actuating mechanism may be used to translate stiffening rod 1906 via cable 1905. In one example, cable 1905 may be understood as having an inner cable portion and an outer cable potion, and in the position shown in FIGS. 19A, the cable may include slack in the inner cable shown by arrow 1905a and tension in the outer cable shown by arrow 1905b. When fixation device 14 is implanted, the temporary stiffness and support of a high-stiffness catheter may make assessing mitral regurgitation (MR) or tricuspid regurgitation (TR) difficult. Thus, to properly assess performance, stiffening rod 1906 may be moved to a second position within, or along, inner conduit 86 to transition the delivery device to a flexible state 19S2 to properly assess the operation of fixation device 14. In FIG. 19B, stiffening rod 1906 has been moved to a relatively proximal position within inner conduit 86 to make the distal portion of the inner conduit more flexible and to reduce the support for fixation device 14. This may be done by, for example, providing slack in the outer cable and adding tension to the inner cable to retract the stiffening rod 1906 proximally. In this position, the distal end of inner conduit 86 is more flexible and the fixation device 14 may more freely move with the native leaflets to provide a more accurate assessment of post-implant performance.

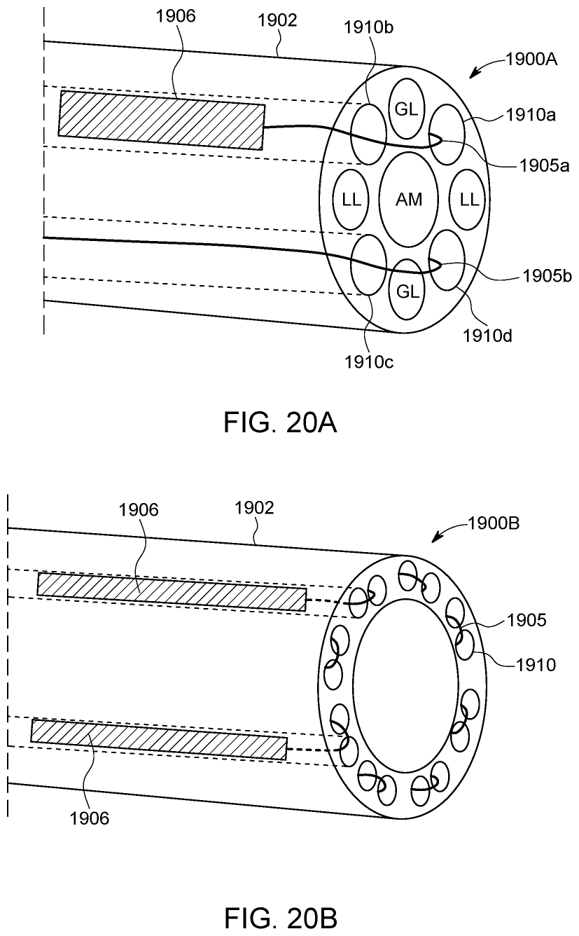

An example of an inner conduit having a body 1902 with multiple stiffening rods 1906 is shown in FIGS. 20A-B. In FIG. 20A, inner conduit 1900A includes a body 1902 having one central actuator mandrel lumen and eight peripheral lumens. The peripheral lumens may include gripper line lumens “GL”, and lock line lumens “LL” as well as two pairs of stiffening rod actuation lumens 1910a-d. A first stiffening rod 1906 having a cable 1905a extending through lumens 1910a,b is shown in the distal position. A second stiffening rod 1906 is disposed within lumen 1910d, but hidden in this illustration, the second stiffening rod 1906 being actuated via cable 1905b that extends through lumens 1910c,d. In this configuration, a balance of stiffness in the conduit is possible, and any number of lumens are possible, provided there is sufficient space. In the shaft shown below, peripheral lumens may be 0.016″ in diameter, and the stiffening rod may be least 0.012″ in diameter. In addition, a design variation where stiffening rods are housed in the wall thickness of a hollow thinner wall catheter shaft is possible as shown in FIG. 20B. In FIG. 20B, a second embodiment of an inner conduit 1900B includes a body 1902 having one central actuator mandrel lumen and eight pairs (for a total of sixteen) of peripheral lumens 1910. Each pair of peripheral lumens 1910 may correspond to a stiffening rod 1906 so that eight stiffening rods are circumferentially disposed about body 1902. In one example, a stiffening rod 1906 is disposed in alternating peripheral lumens 1910. As will be readily understood by those skilled in the art, the examples shown in FIG. 20B may apply to any hollowed catheter system using circumferentially positioned stiffening rods that are housed within the wall thickness, with pull wires being located just circumferentially adjacent to the stiffening rod position. In this manner, hollow catheter components such as the steerable sleeve, or the outer sheath, or both may be stiffened.

In FIGS. 20A-B, the user may transition the system between the responsive and stable configuration with a distally advanced stiffening rod(s) and less stable configuration with a proximally retracted stiffening rod(s), by actuating the stiffening rod(s). This may be accomplished by rotating a pulley knob on the device handle, which tensions the cable on the distal side or proximal side of a stiffening shaft. As tension is applied in one direction, wire is fed from the opposite direction, enabling the exact position of the stiffening rod to be controlled by the user. Stiff and flexible scenarios are shown in FIGS. 19A-B for implant delivery catheter. Similar stiff and flexible scenarios are possible using stiffening rods in the outer sheath or the steerable sleeve as well (e.g., the stiffening rods may be disposed within or abut any one of the elements of the delivery device). In addition, any combination of these elements may have moveable stiffening rod features.

In some examples, for device delivery through venous anatomy, the flexible configuration is preferred as this results in the lowest possible profile and the most flexibility. In some examples, for steering and implant positioning, the stiff configuration is favorable as it increases stability, allowing the user to steer the system more accurately. In some examples, for device deployment and testing, the stiffening rods may be retracted to purposefully reduce the distal stability of the system. This “flexible” state of the catheter does not apply significant forces to the valve being repaired and therefore provides a better simulation of the post-deployment valve repair state even when the fixation device is still attached.

For simplicity, small numbers of longitudinal stiffening rods were shown in the figures in this disclosure. While this was shown for clarity, any number of rods may be used and it may be advantageous to have at least three stiffening rods disposed 120 degrees apart for balance, and multiples of three or four to balance stiffness within the delivery system cross-section. Additionally, the stiffening rods may be made of a number of materials, and may comprise a metal (e.g., stainless steel, cobalt chrome, titanium, tungsten, etc.), or a suitable polymer with a high durometer or modulus, or a glass-filled composite such as Nylon. Radiopaque metals may also be used so that the user can confirm the position of stiffening rods via fluoroscopy or X-Ray imaging during a procedure. Though lumens aligned longitudinally were shown in this disclosure, alternatives are possible such as helically-aligned lumens within the catheter shaft, or within the catheter tubing wall thickness. In some examples, shafts may be made of traditional extrusion materials, may include braiding and/or outer jackets, and may have any number of major and minor lumens. In some examples, pairs of minor lumens positioned radially around the outer perimeter of the shaft or tubing can house a stiffening rod made of any typical stiff metals, and cables may be made of polyester suture material, stainless steel cabling, tungsten cabling, or other materials having very low bending stiffness. Using these configurations, the user may dynamically adjust the position of any one stiffening rod or all of the stiffening rods, depending on the knob attachment(s) on the device handle. In some examples, instead of cabling and pulleys, an alternate system is possible using pneumatic pressure to push or pull stiffening rods within the minor lumens. For this embodiment, distal lumen holes may be joined for pressure continuity.

### D. Tight and Loose Interfaces for Variable Stiffness Delivery System

In another embodiment, variable stiffness of a delivery system may be achieved by providing one or more retractable support sheaths that control the diametric slop at catheter interfaces between the outer sheath and the steerable sleeve, between the steerable sleeve and the inner conduit, or combinations of both. In these embodiments, changes in flexibility in the delivery system is provided by sliding stiffening sheaths that have the property of reducing or increasing the gaps (diametric “slop”) between the catheters, which provides additional changes in the flexibility of the catheter system.

In typical construction of catheters, gaps between catheter layers are minimized to less than 20% of the diameter of the shafts and/or hollow tubular members, and diametric gaps are preferably less than 10% to ensure proper fit. Conversely, a multi-layer catheter may be formed with large gaps intentionally present between the catheter layers. In the present disclosure, gaps between layers may be intentionally increased to create extra slop (i.e., diametric gaps) between the components. For example, gaps greater than 25% between layers of the catheter are possible.

Significant gaps or “slop” may result in a more flexible catheter. Conversely, to create a more responsive and stable tight fit scenario, sliding support sheaths are incorporated in the catheter design that have a slender thickness along their length and a focally increased thickness at the distal end. With this support sheath feature, a catheter-to-catheter tight fit configuration occurs when a support catheter is retracted to nest its distal “thick” region back snugly into the space between catheters, which minimizes slop between catheters. The loose fit configuration occurs when a support catheter is advanced to create intentional slop between catheters, and the device can be transitioned between the tight fit and loose fit configurations by simply retracting or advancing the support sheath.

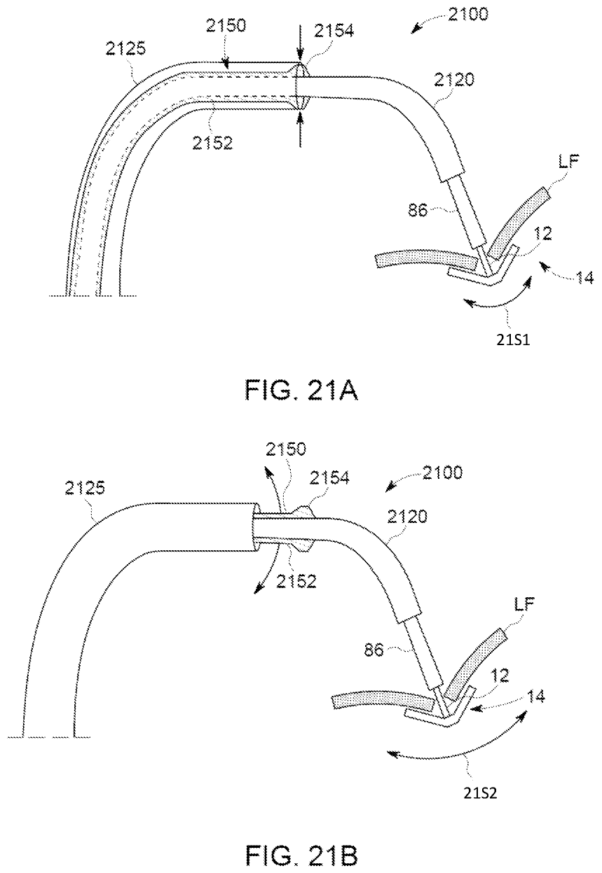

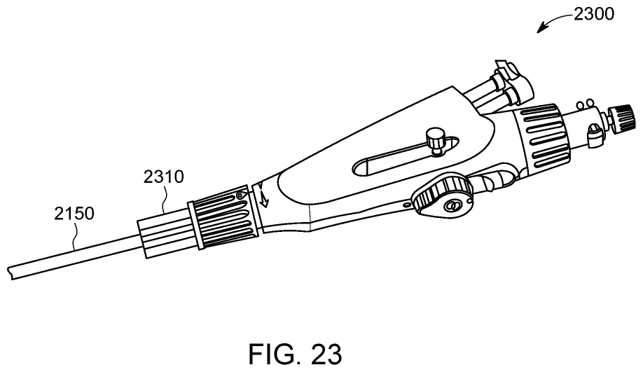

By way of illustration, FIGS. 21A-B show a delivery device 2100 having a shaft 12, an inner conduit 86, a steerable sleeve 2120, and an outer sheath 2125, the delivery device 2100 being coupled to, and capable of delivering, a fixation device 14. In the rigid state 21S1 shown in FIG. 21A, an intermediate support 2150 is disposed in the proximal position, the intermediate support 2150 being disposed between the outer sheath 2125 and the steerable sleeve 2120 to provide additional stiffness for more accurate steering, positioning and/or movement. As shown, intermediate support 2150 may include a main segment 2152 having a first diameter and a flared distal end 2154 having a second diameter larger than the first diameter. In some examples, for flared distal end 2154 to provide significant adjustment of the interface between catheters, it may effectively increase the diameter of main segment 2152 by 20%, or at least 10% and up to 33%. In the rigid state 21S1 shown in FIG. 21A, intermediate support 2150 is disposed between outer sheath 2125 and the steerable sleeve 2120, with flared distal end 2154 being tightly fit between the two elements to increase stiffness. Conversely, in the flexible state 21S2 shown in FIG. 21B, intermediate support 2150 is still disposed between outer sheath 2125 and the steerable sleeve 2120, but has been translated distally to release flared distal end 2154 from contact with outer sheath 2125, creating a looser fit between the elements for more flexibility. As shown in FIG. 23, in some embodiments, a handle 2300 having a knob 2310 or other actuating mechanism may be used to translate intermediate support 2150 directly or indirectly (e.g., via cables) to control the overlap with outer sheath 2125 and/or the gaps between outer sheath 2125 and the steerable sleeve 2120.

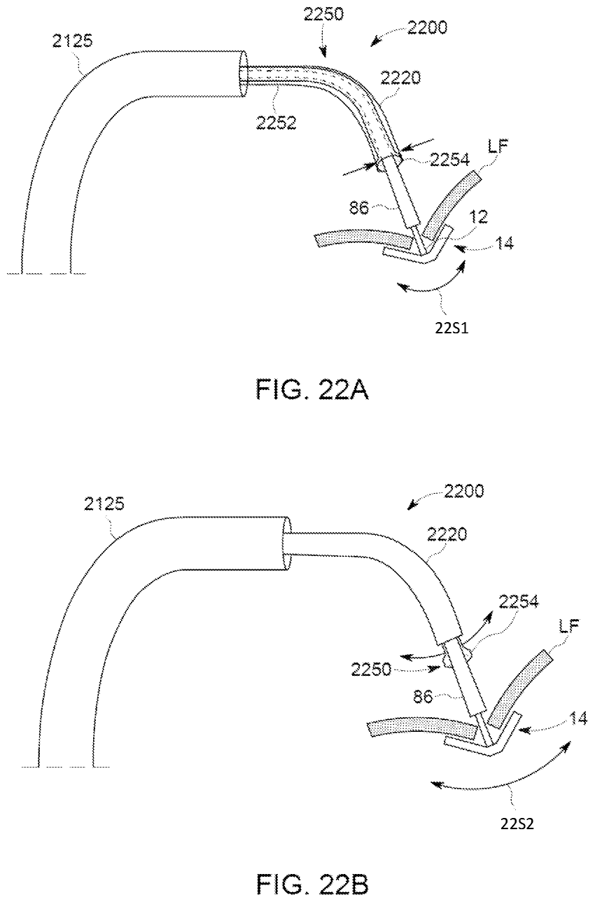

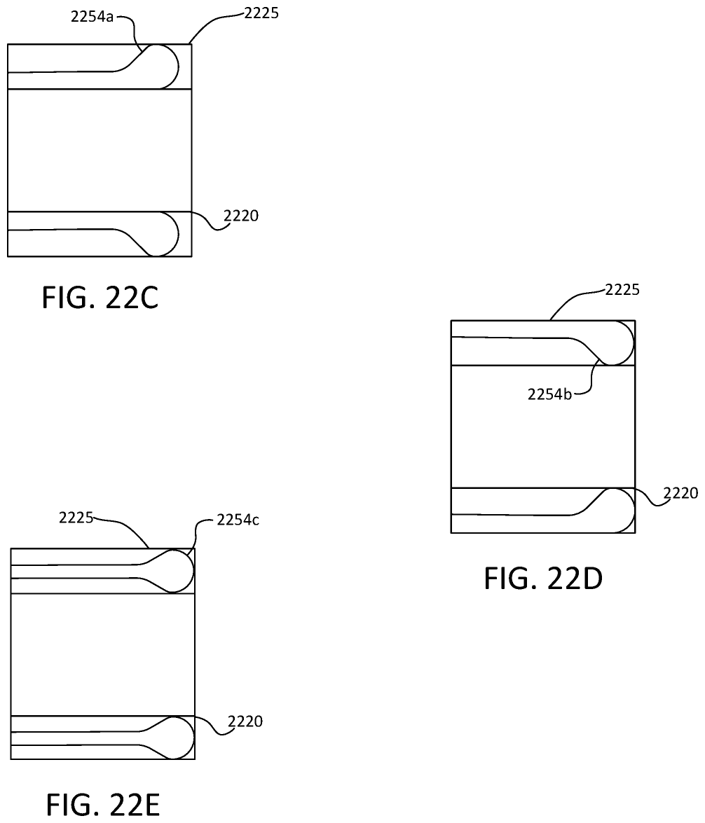

FIGS. 22A-B show one variation of this configuration with a delivery device 2200 having a shaft 12, an inner conduit 86, a steerable sleeve 2220, and an outer sheath 2225, the delivery device 2200 being coupled to, and capable of delivering, a fixation device 14. Delivery device 2200 is also transitionable between multiple states. In the rigid state 22S1 shown in FIG. 22A, an intermediate support 2250 is disposed in the proximal position between the steerable sleeve 2220 and the inner conduit 86 to provide additional stiffness for more accurate steering, positioning and/or movement. As shown, intermediate support 2250 may include a main segment 2252 having a first diameter and a flared distal end 2254 having a second diameter larger than the first diameter. In some examples, for the flared distal end 2254 to provide significant adjustment of the interface between catheters, it may effectively increase the diameter of the main segment 2252 by 20%, or at least 10% and up to 33%. In the rigid state 22S1 shown in FIG. 22A, intermediate support 2250 is disposed between steerable sleeve 2220 and the inner conduit 86, with flared distal end 2254 being tightly fit between the two elements to increase stiffness. Conversely, in the flexible state 22S2 shown in FIG. 22B, intermediate support 2150 is still disposed between steerable sleeve 2220 and the inner conduit 86, but has been translated distally to release flared distal end 2154 from contact with steerable sleeve 2220, creating a looser fit between the elements for more flexibility. Thus, it will be understood that the general principle of having a translatable intermediate member between components to increase or decrease gaps between the elements may be used to provide a stiffer delivery catheter for delivery, and a more flexible catheter in other situations (e.g., fixation device performance testing).

In some examples, for device delivery through venous anatomy, the loose fit configuration may be preferred as this results in the lowest possible profile and the most flexibility. In some examples, for steering and implant positioning, the tight fit configuration may be favorable as it increases stability when the intermediate support is retracted to a snug fit between elements. This snug fit tightens the catheter layer interfaces, reducing the slop between the components, and produces a condition where the delivery system is more stable and can be steered more accurately. In some examples, for device deployment, the intentionally larger gaps present in the “loose state” purposefully reduce the stability of the system. This “loose” state of the delivery device does not apply significant forces to the valve being repaired and therefore provides a better simulation of the post-deployment valve repair state even when the fixation device is still attached. Thus, the stiffness of the catheter may be varied with each step according to need.

In some embodiments, the intermediate support 2250 is circumferentially disposed about, and tightly fit with an inner member and have a snug fit region of increased thickness. Intermediate support 2250 may comprise a laser-cut hypotube, a polymeric extrusion, a braid reinforced coil with thin (loose fit) and thick (snug fit) encapsulated regions with a relatively high durometer Pebax (i.e., 72D), and/or a glass reinforced Nylon 11 or Nylon 12. Certain portions of the delivery catheter (e.g., intermediate support 2250) may further comprise a PTFE liner, or alternatively, a hydrophilic coating on its inner and/or outer diameter to facility smooth translation between the two states.

As previously described, a handle having a knob may be used to actuate the intermediate support. In one embodiment, the handle control to advance or retract the sheath is a lever or rotating knob that advances or retracts the support sheath. In an alternate embodiment, the handle control to advance and retract the sheath is a coaxial rotating screw that forcibly and controllably advances or retracts the sheath. In some embodiments, the intermediate support may default to a “tight configuration” absent actuation by the user. Alternatively, the intermediate support may default to a “loose configuration” absent actuation by the user.

Variations are possible. For example, in some embodiments, the intermediate support rides on the outer diameter of the outer element (e.g., steerable sleeve) and has an increased thickness at its distal end, where the distal end is thickened in the outward direction toward the inner element inner diameter (e.g., outer sheath) (FIG. 22C). In some embodiments, the intermediate support is attached to the outer element inner diameter (e.g., outer sheath) and has an increased thickness at its distal end, where the distal end is thickened in the inward direction toward the inner element (e.g., steerable sleeve) (FIG. 22D). The intermediate support may also be thickened at the distal end in both inner and outer directions (FIG. 22E). In some embodiments, the intermediate support is keyed and controls the orientation of curves between the outer sheath and the steerable sleeve. In some embodiments, retracting and advancing the intermediate support engages or disengages keying, where the leading edge of the outer sheath has a taper to allow the keyed region to re-align when pulled back. In some embodiments, the intermediate support is disposed between the steerable sleeve and the inner conduit. In some embodiments, multiple intermediate supports are present between different layers (e.g., a first intermediate support between the outer sheath and the steerable sleeve, and a second intermediate support between the steerable sleeve and the inner conduit). In some embodiments, advancing the intermediate support allows for proximal atrial pressure measurement through the wider gaps between the layers.

### E. Retractable Support Sheath

In some example, in order to preserve the navigation and delivery benefits of a rigid delivery catheter, while overcoming the deployment and MR/TR assessment challenges, a dual delivery catheter system may be used which comprises a flexible inner member, and a rigid outer member, where the outer member provides the rigidity needed to navigate and delivery the implant to the valve, but can be retracted prior to deployment, exposing the flexible delivery catheter, and allowing for a more accurate assessment of MR/TR prior to implant release.

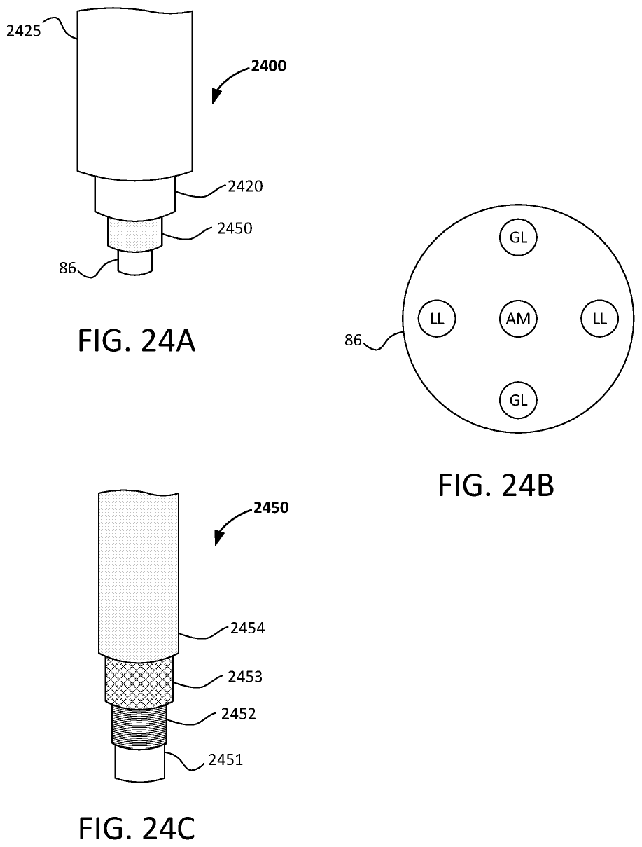

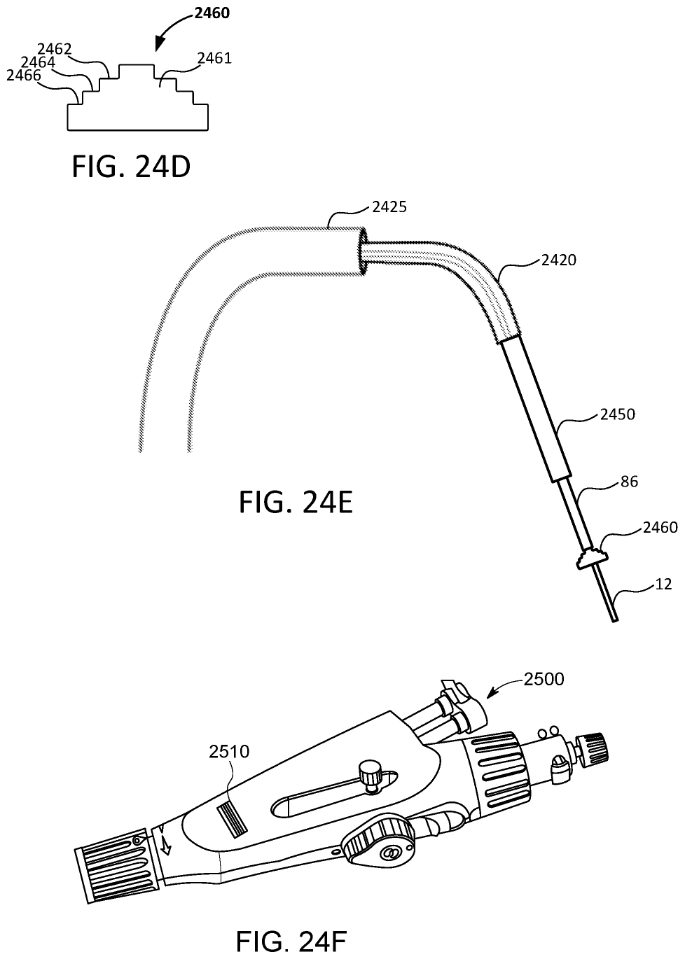

FIG. 24A illustrates layers of a delivery device 2400 according to one embodiment of the present disclosure. As shown in FIG. 24A, delivery device 2400 generally includes an outer sheath 2425, a steerable sleeve 2420, a support sheath 2450, and an inner conduit 86. The outer sheath 2424, a steerable sleeve 2420, a support sheath 2450, and inner conduit 86 may be translatable relative to one another.

As shown in FIG. 24B, inner conduit 86 may be constructed from a low-durometer multi-lumen extrusion (e.g., Pebax 35D or similar), and include a plurality of lumens, which may include lock line lumens “LL”, gripper line lumens “GL”, and an actuator mandrel lumen “AM”. Traditional inner conduits may include a compression coil in the central lumen, but this may be eliminated, which would reduce rigidity and allow for an outer diameter reduction of the inner conduit. Traditional inner conduits 86 are typically about 0.125 in diameter. In some examples the reduced outer diameter of the inner conduit is between 0.070 and 0.090.

Turning to FIG. 24C, it will be appreciated that the resulting reduction in the outer diameter of the inner conduit 86 from eliminating the inner compression coil may allow for the inclusion of support sheath 2450 between inner conduit 86 and steerable sleeve 2420 without increasing the overall diameter of a delivery device. In some examples, the nested support sheath 2450 and inner conduit 86 would be no large in diameter than a traditional inner conduit 86 (e.g., 0.125 in diameter). Support sheath 2550 may provide rigidity to offset the loss in rigidity in inner conduit 86. In some examples, support sheath 2450 may include an inner liner layer 2451 (e.g., PTFE), a second layer 2452 (e.g., coiled stainless steel wire), a third layer 2453 (e.g., braided stainless steel wire), and an outer layer 2454, such as a relatively high durometer covering (e.g., Pebax 72D, Nylon 11, Nylon 12, etc.). In some examples, inner conduit 86 comprises a hydrophilic coating on its outer diameter to facility smooth translation between it and support sheath 2550. In some examples, support sheath 2550 may include a hydrophilic coating on its outer diameter to facilitate translation between it and steerable sleeve 2420.

In addition to support sheath 2450, a delivery catheter may also include a retraction ring 2460 having body 2461 with a number of steps as shown in FIG. 24D. Specifically, retraction ring 2460 may include a first face 2462 for bonding to inner conduit 86, a second face 2464 to serve as a stop for the support sheath 2450, and a third face 2466 to serve as a stop for steerable sleeve 2420. Retraction ring 2460 may be affixed to the distal end of the inner conduit 86 to prevent retraction of the inner conduit 86 into steerable sleeve 2420, and may be radiopaque for visualization. Retraction ring 2460 may also perform a secondary function of preventing the inner conduit 86 from being retracted into support sheath 2450. FIG. 25A illustrates the use of the retraction ring 2460 as it is bonded to inner conduit 86.

As shown in FIG. 24F, in some embodiments, a handle 2500 having a lever 2510 or other actuating mechanism may be used to translate support sheath 2450 directly or indirectly (e.g., via cables) to control the overlap with outer sheath 2425, the steerable sleeve 2420 and/or the inner conduit 86. This may allow the interventionalist to retract support sheath 2450 prior to deployment for a more accurate assessment of MR/TR. Additionally, lever 2510 may allow for advancement of support sheath 2450 if the interventionalist is not satisfied with the amount of MR/TR reduction and chooses to regrasp leaflets. Optionally, a seal (e.g., an o-ring or gasket) (not shown) may be added to the interface between the outer diameter of the lever 2510, and handle 2500 to maintain hemostasis.

It is to be understood that the embodiments described herein are merely illustrative of the principles and applications of the present disclosure. For example, a system may include any number of peripheral lumens or any number of transitions between helical and straight paths. Additionally, a system may include both helical and non-helical paths, including straight paths. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. An interventional tool, comprising:
an outer sheath;
a steerable sleeve disposed within the outer sheath and translatable relative thereto;
an inner conduit disposed within the steerable sleeve and translatable relative thereto; and
a stiffness-varying element disposed within the outer sheath and configured and arranged to transition the interventional tool between a first state having a first stiffness and a second state having a second stiffness, the first stiffness being greater than the second stiffness.

2. The interventional tool of claim 1, wherein the stiffness-varying element comprises at least one stiffening rod translatable within the interventional tool.

3. The interventional tool of claim 2, wherein the at least one stiffening rod is disposed within the inner conduit and configured to translate from a distal position in the first state, and a proximal position in the second state.

4. The interventional tool of claim 2, wherein the at least one stiffening rod is coupled to an actuating cable.

5. The interventional tool of claim 2, wherein the at least one stiffening rod comprises multiple stiffening rods that are independently actuatable.

6. The interventional tool of claim 1, wherein the stiffness-varying element comprises an intermediate support having a main segment with a first diameter, and a flared distal end with a second diameter larger than the first diameter.

7. The interventional tool of claim 6, wherein the intermediate support is disposed between the outer sheath and the steerable sleeve, and configured to translate between a first position where the flared distal end, the outer sheath and the steerable sleeve are friction fit with one another, and a second position where the flared distal end, the outer sheath and the steerable sleeve are not friction fit with one another.

8. The interventional tool of claim 6, wherein the intermediate support is disposed between the outer sheath and the steerable sleeve, and configured to translate between a first position where the flared distal end is directly in contact with the outer sheath, and a second position where the flared distal end spaced from the outer sheath.

9. The interventional tool of claim 6, wherein the intermediate support is disposed between the steerable sleeve and the inner conduit, and configured to translate between a first position where the flared distal end, the steerable sleeve and the inner conduit are friction fit with one another, and a second position where the flared distal end, the steerable sleeve and the inner conduit are not friction fit with one another.

10. The interventional tool of claim 6, wherein the intermediate support is disposed between the steerable sleeve and the inner conduit, and configured to translate between a first position where the flared distal end is directly in contact with the steerable sleeve, and a second position where the flared distal end is spaced from the steerable sleeve.

11. The interventional tool of claim 1, wherein the stiffness-varying element comprises a support sheath disposed between the inner conduit and the steerable sleeve, the support sheath comprising at least two layers.

12. The interventional tool of claim 11, wherein the support sheath comprises at least an inner liner layer, a coiled metal layer, a braided layer and an outer covering.

13. The interventional tool of claim 11, wherein the support sheath comprises at least a braided wire and a coiled wire.

14. A method of actuating a medical device, comprising:
providing an interventional tool including an outer sheath, a steerable sleeve disposed within the outer sheath and translatable relative thereto, an inner conduit disposed within the steerable sleeve and translatable relative thereto, and a stiffness-varying element disposed within the outer sheath; and
transitioning the interventional tool between a first state having a first stiffness and a second state having a second stiffness, the first stiffness being greater than the second stiffness.

15. The method of claim 14, wherein transitioning the interventional tool comprises actuating the stiffness-varying element.

16. The method of claim 14, wherein transitioning the interventional tool comprises translating the stiffness-varying element relative to at least one of the outer sheath, the steerable sleeve and the inner conduit.

17. The method of claim 14, further comprising the step of navigating the interventional tool to a heart valve in a first state to deliver a prosthetic implant, and transitioning the interventional tool to the second state to test performance of the prosthetic implant.

18. The method of claim 17, wherein the prosthetic implant is a leaflet fixation device, and wherein transitioning the interventional tool to the second state to test performance of the prosthetic implant comprises assessing the heart valve for regurgitation while the interventional tool is in the second state.
